(19) United States
(12) Patent Application Publication
(10) Pub. No.: US 2003/0014065 A1
(43) Pub. Date: Jan. 16, 2003

(54) Endoscopic instrument

(76) Inventor: J?ouml;rgen Osterlind, (US)

Correspondence Address: BROWDY AND NEIMARK, P.L.L.C., 624 NINTH STREET, NW, SUITE 300, WASHINGTON, DC, 20001-5303, US

(21) Appl. No.: 10/181,717

(22) PCT Filed: Oct. 5, 2001
(86) PCT No.: PCT/SE01/02160

(30) Foreign Application Priority Data
Oct. 5, 2000 (SE) 0003606

(51) Int. Cl. A61B 17/28

(52) U.S. Cl. 606/167; 606/207; 606/205

(57) ABSTRACT

An endoscopic surgical instrument comprises an elongate main body (1) defining a longitudinal axis and having a distal end side (2) and a proximal end side (3). Surgically operating means (4, 5) are disposed on the distal end side (2) of the main body (1), and manipulating means are disposed on the proximal end side (3) of the main body (1) and adapted to effect remote control of the surgically operating means (4, 5). Elongate transmitting means are adapted to transmit operation of the manipulating means to the surgically operating means (4, 5). The surgically operating means comprises a first surgically operating means (4) comprising a pair of forceps having a gripping function in a first plane, and a second surgically operating means (5) comprising a device selected from a group consisting of a lifting device, a pulling device, and a cutting device.

The present invention generally relates to an instrument for use in minimally invasive surgical procedures. More specifically, it relates to an endoscopic surgical instrument, which comprises an elongate main body defining a longitudinal axis and having a distal end side and a proximal end side, surgically operating means disposed on the distal end side of the main body, manipulating means disposed on the proximal end side of the main body and adapted to effect remote control of the surgically operating means, and elongate transmitting means adapted to transmit operation of the manipulating means to the surgically operating means.

Minimally invasive surgery, i.e. endoscopic surgery, and instruments for use in such surgery are well known in the art. Generally, minimally invasive surgery involves the use of instruments with relatively long narrow operation portions, which may be deployed through small incisions in the skin. Trocars may be inserted into the incisions in order to protect the surrounding tissue. During a surgery, these trocars may be used for insertion of endoscopic instruments, such as a pair of forceps and a pair of scissors. A miniature camera may also be inserted for observation of the surgery on a screen.

Thus, endoscopic procedures gain access to the inside of an anatomical cavity by using an implement, such as a trocar, a cannula, or a needle having a sharpened point to pierce or puncture the bodily tissues, muscles, membranes, or the like, which may form a portion of or surround the cavity wall.

Since the area in which the surgeon must perform procedures incorporating endoscopic surgical instrumentation is smaller than that normally encountered, surgical instrumentation must take this factor into consideration.

Further, when several instruments are required for a minimal invasive surgery it is essential that these instruments be designed so as to facilitate co-ordination of the operation thereof.

### SUMMARY OF THE INVENTION

An object of the present invention is to provide an endoscopic instrument, which reduces the space requirements of an endoscopic surgery.

Another object of the present invention is to facilitate manipulating when two endoscopic instruments are involved in performing a specific surgery operation.

Still another object of the present invention is to provide an endoscopic instrument adapted for introducing a graft connector into a blood vessel in a minimally invasive surgery operation.

To achieve the objects described above, this invention provides an endoscopic surgical instrument of the type described by way of introduction, wherein the surgically operating means comprises a first surgically operating means comprising a pair of forceps having a gripping function in a first plane, and a second surgically operating means comprising a device selected from a group consisting of a lifting device, a pulling device, and a cutting device.

In a preferred embodiment, the second surgically operating means comprises a lifter having a lever pivotable in a second plane perpendicular to the first plane.

In the preferred embodiment, the lever of the lifter is bifurcated and has knobs extending towards each other. Thus, the preferred embodiment is especially adapted for use when introducing a graft connector as disclosed in International Patent Application No. PCT/SE00/01610. In that case, the first plane is perpendicular to the longitudinal axis of the main body.

Preferably, the manipulating means comprises a cylindrical actuator, which is slidable along the main body and is connected to the elongate transmitting means to transmit operation to the second surgically operating means. Also, the main body may comprise a tube of circular cross-section and rotatable about its longitudinal axis, and the cylindrical actuator then has a through bore receiving the tube.

Further, the tube may have a longitudinal slit and the elongate transmitting means may comprise a bar having a projection connected to the cylindrical actuator via the longitudinal slit.

### BRIEF DESCRIPTION OF THE DRAWINGS

FIG. 1 is an elevational view of one embodiment of an endoscopic instrument according to the present invention;

FIG. 2 is a top plan view of the endoscopic instrument shown in FIG.1;

FIG. 3 is an enlarged sectional view along lines III-III in FIG. 2 and illustrates the endoscopic instrument in a first state;

FIG. 4 corresponds to FIG. 3 but illustrates the endoscopic instrument in a second state;

FIGS. 5, 6 and 7 are enlarged bottom, elevational and top views of a distal end part of the endoscopic instrument of FIGS. 1-4 shown in a closed state;

FIGS. 8, 9 and 10 correspond to FIGS. 5, 6 and 7, respectively, but illustrate an open state;

FIG. 11 is a perspective view illustrating the endoscopic instrument of FIGS. 1-10 and a graft connector gripped thereby.

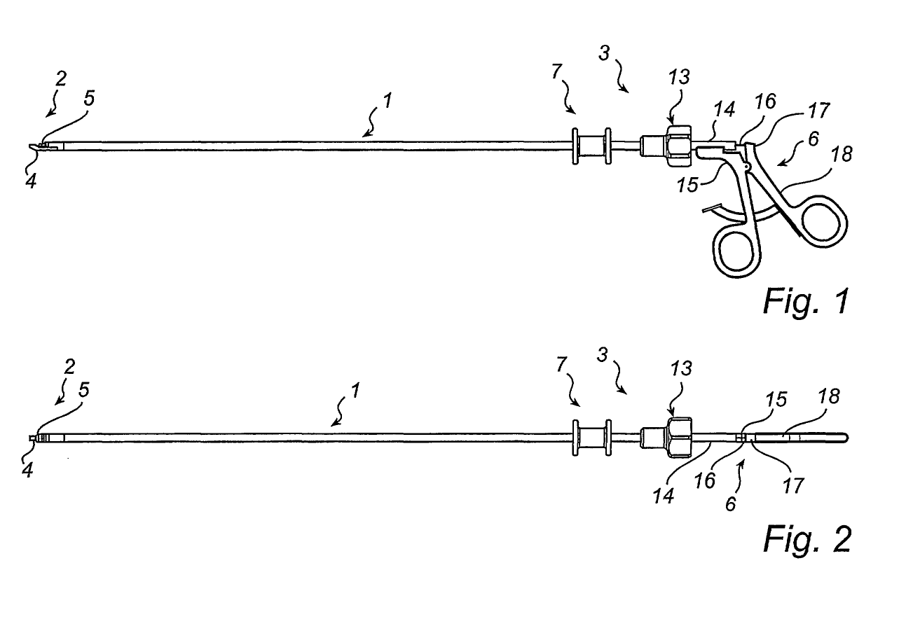

### DESCRIPTION OF A PREFERRED EMBODIMENT

Referring to the drawings, an embodiment of an endoscopic surgical instrument comprises an elongate main body 1, which defines a longitudinal axis and has a distal end side 2 and a proximal end side 3. A distal end part is disposed on the distal end side 2 of said main body 1 and is provided with a first and a second surgically operating means 4 and 5. A manipulating part is disposed on the proximal end part 3 of said main body 1 and is provided with a first and a second manipulating means 6 and 7 for remote control of said surgically operating means 4 and 5, respectively.

More precisely, the main body 1 comprises a tube 8 having at the distal end side 2 a pair of forceps constituting said first surgically operating means 4 and a lifter constituting said second surgically operating means 5.

The pair of forceps 4 has two jaws 9, 10 pivotable about an axis 11 in response to translational movements by a first manoeuvring bar 12 constituting an elongate transmitting member adapted.to transmit operation of the first manipulating means 6 to the first surgically operating means 4. This manoeuvring is conventional and uses the bar 12 for remote control of the pair of forceps 4, i.e. opening and closing of the jaws 9, 10.

The tube 8 is fixed to a hub 13 which is rotatable on a shaft 14 fixed to a handle body 15. The first manoeuvring bar 12 extends coaxially through the proximal part of the,tube 8 and coaxially through the hub,13 and the shaft 14. A free proximal end 16 of the first manoeuvring bar 12 engages an end of a lever 17 on a handle actuator 18 pivotable on the handle body 15.

A clockwise pivoting of the handle 18 will retract the first manoeuvring bar 12 towards the proximal end side 3 and move the jaws 9, 10 toward a closed state, while an anti-clockwise pivoting of the handle 18 will push the first manoeuvring bar 12 towards the distal end side 2 and move the jaws 9, 10 toward an open state of a maximum of about 45° between the jaws 9, 10.

The lifter 5 is a lever pivotable on an axis 19 between a closed position aligned with the longitudinal axis of the main body 1 and an open position at a maximum angle of about 45° relative to the longitudinal axis of the main body 1. The lifter 5 pivots in response to translational movements by a second manoeuvring bar 20 constituting an elongate transmitting member adapted to transmit operation of the manipulating means 7 to the second surgically operating means 5, i.e. the lifter.

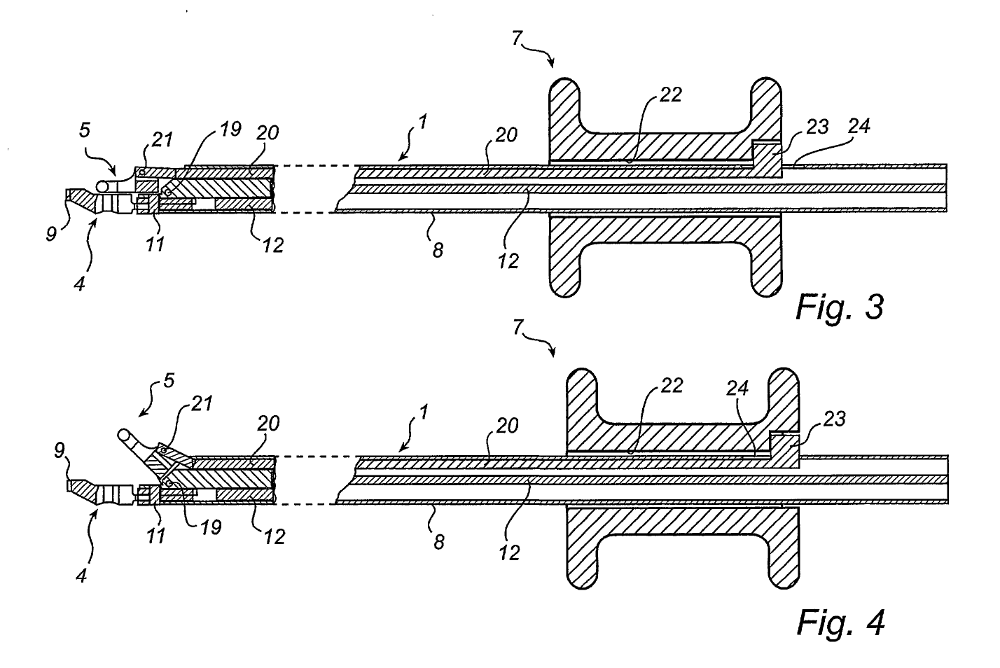

The distal end of the second bar 20 is fixed to the lifter 5 at a point 21 distal to the axis 19, as best seen in FIGS. 3 and 4, and the proximal end of the second bar 20 engages a cylindrical actuator constituting the second manipulating means 7. The cylindrical actuator 7 has a through bore 22 of substantially the same diameter as the tube 7 so as to be slidable thereon. The cylindrical actuator 7 is fixed to a radial projection 23 at a proximal end of the second manoeuvring bar 20. This projection will slide in a longitudinal slit 24 in the tube 8 when the actuator 7 is made to slide on the tube 8. Obviously, the lever of the lifter 5 will assume its closed position aligned with the longitudinal axis of the main body 1 when the actuator 7 is pushed to its maximum distal position on the tube 8, and the lever of the lifter 5 will assume its open position forming an angle of about 45° relative to the longitudinal axis of the main body 1 when the actuator 7 is retracted to its maximum proximal position on the tube 8.

The surgically operating means 4 and 5 at the distal end side 2 may be oriented in any direction in a plane perpendicular to the longitudinal axis of the main body 1 by rotating the hub 13 on the shaft 14.

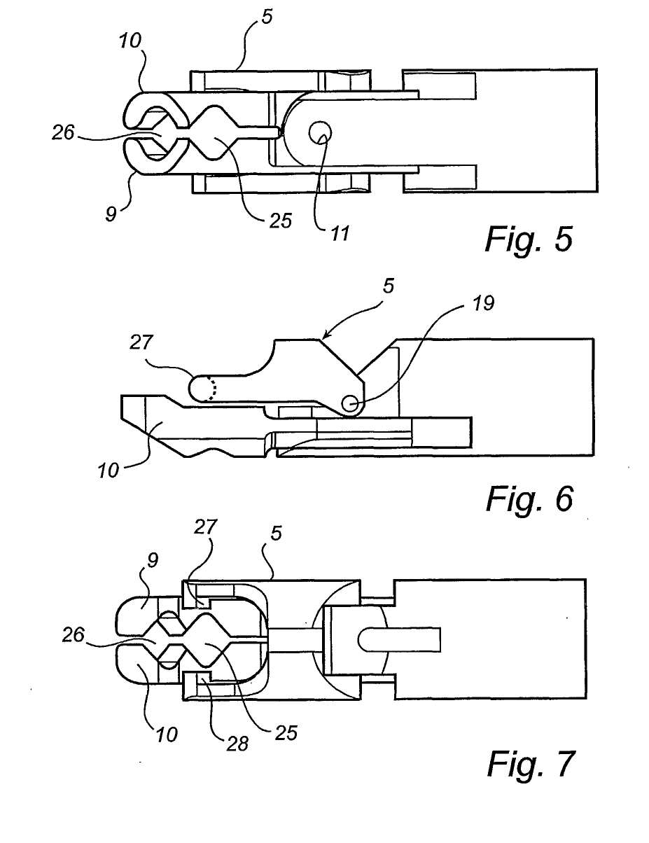

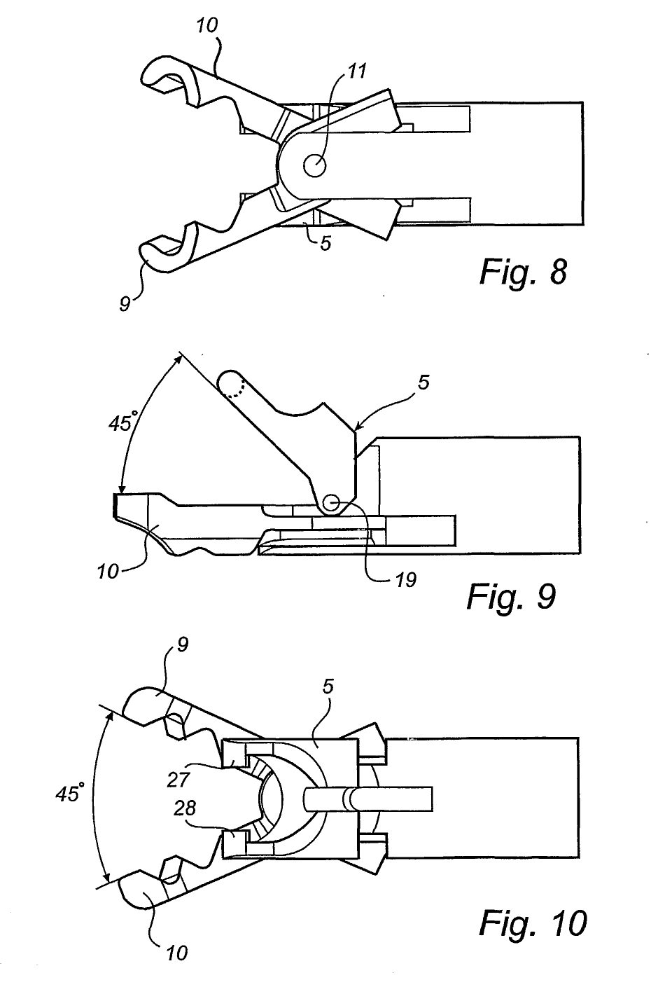

As best illustrated in FIGS. 5 and 7, the jaws 9, 10 of the pair of forceps 5 each have two indentations forming two channels 25, 26 of non-circular cross-section in the closed state of the jaws 9, 10. The channel 25 is perpendicular to the longitudinal axis of the main body 1, and the channel 26 forms an angle of about 60° relative to the longitudinal axis of the main body 1.

As best illustrated in FIGS. 7 and 10, the lever of the lifter 5 is bifurcated so as to form two free distal ends having knobs 27, 28 directed towards each other.

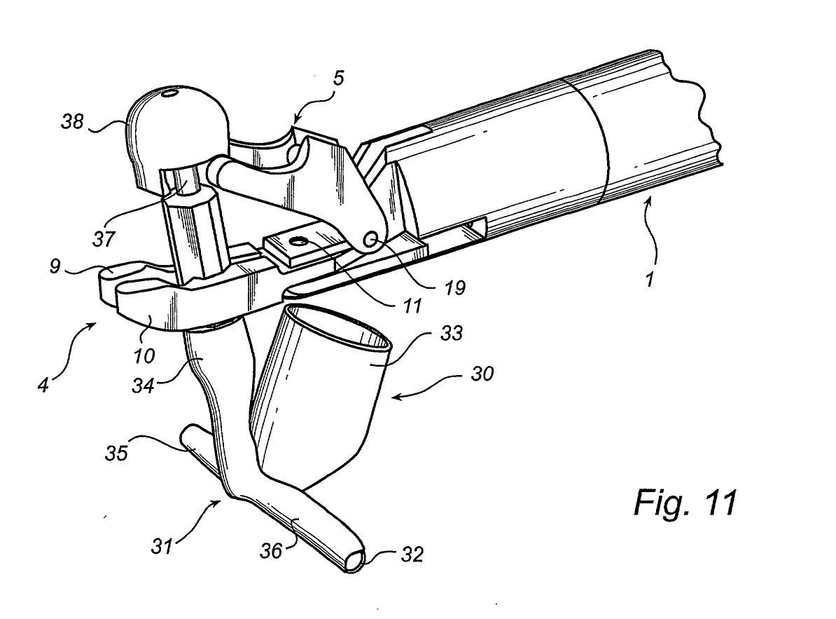

The above-described embodiment of an endoscopic instrument according to the present invention is especially designed for use in a surgery involving the introducing of a graft connector of the type disclosed in International Patent Application No. PCT/SE00/01610. Such use is illustrated in FIG. 11, where a graft connector 30 is held by an introducer 31. The graft connector 30 comprises a sleeve 32 and a collar 33 extending perpendicular from the sleeve 32. The introducer 31 is a T-shaped element having an octagonal shaft 34 and two oppositely directed arms 35, 36, the sleeve 32 being fixed along these arms 35, 36. A needle 37 coaxially positioned in the shaft 34 will release the graft connector 30 from the introducer 31 when retracted in the shaft 34. At its end projecting from the shaft 34, the needle 37 has a cap 38 designed for engagement with the knobs 27, 28 of the lifter 5.

A minimally invasive surgery may involve introducing, by means of the above-described endoscopic instrument the sleeve 32 of the graft connector 30 into an artery downstream of a blockage in the artery in order to bypass the blockage. When performing such a surgery, the endoscopic instrument according to the present invention may be used in the way illustrated in FIG. 11. Thus, the shaft 34 of the introducer 31 first is grasped in the channel 25 formed by the jaws 9, 10 in their closed state. Then, the endoscopic instrument is manipulated such that the sleeve 32 is introduced through an incision made in the artery. Thereafter, the lever 5 is lifted to its open state, whereby the cap 38 and the needle 37 are retracted from the shaft 34, and the introducer 31 is released from the graft connector 30. Finally, the introducer 31 is retracted from the artery and pulled out of the body being the object of the surgery.

As is evident to the man skilled in the art, the above-described embodiment of an endoscopic instrument may be modified in several aspects within the scope of the present invention. Thus, the instrument may be used for making an incision in any body vessel by providing the lever/lifter 5 with an appropriate edge. In such case, a vessel such as an artery may be grasped using the jaws 9, 10 of the pair of forceps and then activating the lever/lifter 5 preferably from its open to its closed state, whereby the edge of the lifter will cut a longitudinal opening in the vessel. Alternatively, the lever 5 could function as a pair of scissors.

As a further modification, the lifter 5 could be a direct extension of the second bar 20, whereby a movement of the cylindrical actuator 7 would result in a corresponding longitudinal movement of the lifter 5. This modification could be combined with such a modification of the jaws 9, 10 that they would grasp the shaft 34 of the introducer 31 in a position where the shaft 34 is aligned with the longitudinal axis of the main body 1.

1. An endoscopic surgical instrument, comprising
an elongate main body (1) defining a longitudinal axis and having a distal end side (2) and a proximal end side (3),
surgically operating means (4, 5) disposed on the distal end side (2) of the main body (1),
manipulating means disposed on the proximal end side (3) of the main body (1) and adapted to effect remote control of the surgically operating means (4, 5),
elongate transmitting means adapted to transmit operation of the manipulating means to the surgically operating means (4, 5), wherein
the surgically operating means comprises
a first surgically operating means (4) comprising a pair of forceps having a gripping function in a first plane, and
a second surgically operating means (5) comprising a device selected from a group consisting of a lifting device, a pulling device, and a cutting device.

2. An endoscopic surgical instrument as claimed in claim 1, wherein the second surgically operating means (5) comprises a lifter having a lever pivotable in a second plane perpendicular to the first plane.

3. An endoscopic surgical instrument as claimed in claim 2, wherein the first plane is parallel to the longitudinal axis of the main body (1).

4. An endoscopic surgical instrument as claimed in claim 2 or 3, wherein the lever is bifurcated having knobs (27, 28) extending towards each other.

5. An endoscopic surgical instrument as claimed in claim 1, wherein the second surgically operating means (5) comprises a lifter translational in a direction parallel to the longitudinal axis of the main body (1).

6. An endoscopic surgical instrument as claimed in claim 3, wherein the first plane is perpendicular to the longitudinal axis of the main body (1).

7. An endoscopic surgical instrument as claimed in any one of claims 1-6, wherein the manipulating means comprises an actuator (7), which is slidable along the main body (1) and is connected to the elongate transmitting means to transmit operation to the second surgically operating means (5).

8. An endoscopic surgical instrument as claimed in claim 7, wherein the main body (1) comprises a tube (8) rotatable about its longitudinal axis and wherein the actuator (7) is cylindrical and has a through bore (22) receiving the tube (8).

9. An endoscopic surgical instrument as claimed in claim 8, wherein the tube (8) comprises a longitudinal slit (24) and wherein the elongate transmitting means comprises a bar (20) having a projection (23) connected to the actuator (7) via the longitudinal slit (24).

10. An endoscopic surgical instrument as claimed in any one of claims 1-9, wherein the pair of forceps (4) comprises two jaws (9, 10) having indentations forming at least one channel (25, 26) of non-circular cross-section.

11. An endoscopic surgical instrument as claimed in any one of claims 1-10, wherein the manipulating means comprises a handle actuator (8) having a lever (17) connected to the elongate transmitting means to transmit operation to the-first surgically operating means (4).
